SEQUENCE LISTING (3 sequences)

SEQ ID NO: 1 - RNA, 30 nt, Artificial Sequence
  NH2 (CH2)6 30-mer RNA
acaugcaaacacacaugaacagucagccca

SEQ ID NO: 2 - DNA, 30 bp, Artificial Sequence
  NH2 (CH2)6 30-mer complementary DNA
tgggctgactgttcatgtgtgtttgcatgt

SEQ ID NO: 3 - DNA, 20 bp, Artificial Sequence
  Forward primer amplifying the cDNA corresponding to the nucleotide sequence from 1,346 and 2,147 of mouse Pax6 mRNA
tctaatcgaagggccaaatg

(19) United States
(12) Patent Application Publication
(10) Pub. No.: US 2012/0237927 A1
(43) Pub. Date: Sep. 20, 2012

(54) Method of Mapping of mRNA Distribution With Atomic Force Microscopy Comprising Dendron

(76) Inventors: Joon Won Park, Pohang (KR); Yu Jin Jung, Busan (KR); Yu Shin Park, Pohang (KR); Hong Gil Nam, Pohang (KR)

(21) Appl. No.: 13/309,552

(22) Filed: Dec. 1, 2011

Related U.S. Application Data
(63) Continuation of application No. 12/416,033, filed on Mar. 31, 2009, now abandoned.
Application No. 12/416,033 is a continuation-in-part of application No. 11/673,732, filed on Feb. 12, 2007.
Application No. 11/673,732 is a continuation-in-part of application No. 11/464,481, filed on Aug. 14, 2006, now abandoned.
(60) Provisional application No. 61/041,209, filed on Mar. 31, 2008.
(60) Provisional application No. 60/707,892, filed on Aug. 12, 2005.
(60) Provisional application No. 60/817,608, filed on Jun. 28, 2006.

(51) Int. Cl. C12Q 1/68

(52) U.S. Cl. 435/6.11

(57) ABSTRACT

The present invention relates to a method of mapping of mRNA distribution, comprising the steps of a preparing a probe DNA attached to a apical liner region of the dendron on AFM cantilever where the probe DNA can specifically hybridize a target mRNA and measuring specific adhesive force between the probe DNA and the target mRNA on sectioned tissue at nanometer resolution.

### CROSS REFERENCE TO RELATED APPLICATION

This application is a Continuation of U.S. patent application Ser. No. 12/416,033, filed Mar. 31, 2009, which claims priority benefit of U.S. Provisional Patent Application No. 61/041,209 filed Mar. 31, 2008, the entire content of which is incorporated hereinto by reference. This application is also a Continuation-in-Part application of U.S. patent application Ser. No. 11/673,732 filed Feb. 12, 2007, which is a continuation-in-part of U.S. patent application Ser. No. 11/464,481, filed Aug. 14, 2006, now abandoned, which claims priority benefit of U.S. Provisional Patent Application No. 60/707,892, filed Aug. 12, 2005, and U.S. Provisional Patent Application No. 60/817,608, filed Jun. 28, 2006, the contents of all of which are incorporated by reference herein in their entirety. This application and U.S. patent application Ser. No. 11/673,732 also claim priority benefit of PCT Patent Application No. PCT/KR2005/002651 filed Aug. 12, 2005, which claims priority benefit of U.S. Provisional Patent Application No. 60/601,237, filed Aug. 12, 2004.

### FIELD OF THE INVENTION

The present invention relates to a method of mapping of mRNA distribution, comprising the steps of a preparing a probe DNA attached to a apical liner region of the dendron on AFM cantilever where the probe DNA can specifically hybridize a target mRNA and measuring specific adhesive force between the probe DNA and the target mRNA on sectioned tissue at nanometer resolution.

### BACKGROUND OF THE INVENTION

Differential expression of mRNA in various cell types is a basic regulatory mechanism of cellular and/or tissue differentiation. Intracellular RNA distribution is now recognized as an essential mechanism in the regulation of localized protein expression. Yet, the sensitivity and resolution of current technologies are not sufficient for understanding the molecular level roles of mRNA concentration and distribution. Atomic force microscopy (AFM) permits recognition of proteins by utilizing antigen-antibody or ligand-receptor interactions, which subsequently allow spatial distribution mapping at nanometer resolution.

When measuring molecular interactions with AFM, the way of immobilizing a probe molecule on the AFM tip is a significant feature. Less-controlled immobilization, in terms of specificity, orientation, and spacing, can result in poor detection of target molecules, leading to unwanted nonspecific interactions and/or broad unbinding force distributions.

### SUMMARY OF THE INVENTION

Detection of the cellular and tissue distributions of RNA species is significant in our understanding of the regulatory mechanisms underlying cellular and tissue differentiation.

An atomic force microscope tip modified with the dendron can be successfully used to map the spatial distribution of mRNA on sectioned tissues of an animal. Scanning of the sectioned tissue with a probe DNA attached to the apex of the dendron resulted in detection of the target mRNA on the tissue section, permitting mapping of the mRNA distribution at nanometer resolution. The unprecedented sensitivity and resolution of this process should be applicable to identification of molecular level distribution of various RNAs in a cell.

The presence and location of mRNA molecules in a sectioned tissue can be facilely detected using a DNA probe attached to a dendron-modified AFM tip. This mRNA detection procedure is straightforward once the DNA probe is properly selected and immobilized on a suitably modified AFM tip. The choice of dendron in AFM tip modification was a critical factor. The use of a 27-acid dendron led to successful detection of the mRNA, whereas tips modified with a lower generation dendron (3-acid or 9-acid) led to unsatisfactory results, with frequent nonspecific and multiple rupture events and broad force histograms.

Accordingly, the present invention provides a method of mapping of mRNA distribution, comprising the steps of a preparing a probe DNA attached to a apical liner region of the dendron on AFM cantilever where the probe DNA can specifically hybridize a target mRNA and measuring specific adhesive force between the probe DNA and the target mRNA on sectioned tissue at nanometer resolution.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A is a schematic drawing of the experimental setup employed for the measurement of the interaction force between the 30-mer DNA probe and the complementary 30-mer oligo RNA. The DNA probe complementary to the sequence between nucleotides 1,698-1,727 of the Pax6 mRNA was immobilized on the 27-acid dendron-modified AFM tip. The 30-mer oligo RNA complementary to the DNA probe sequence was immobilized on a 27-acid dendron-modified silicon substrate.

FIG. 1B shows structure of the 27-acid dendron, 9-anthrylmethyl-3-({[tris({[(1-{tris[(2-{[(tris {[2-carboxyethoxy]methyl}methyl)amino]carbonyl}ethoxy)methyl]methyl}amino)carbonyl]-2-ethoxy}methyl)methyl]amino}carbonyl)propylcarbamate. Shown to the right is a schematic diagram of the DNA probe attached to the apex of a dendron on a substrate such as an AFM tip.

FIG. 1C shows structure of Pax6 mRNA. The DNA sequence complementary to the mRNA sequence between nucleotides 1,698-1,727 was used in the synthesis of the DNA probe, 5′—NH₂(CH₂)₆-TGG GCT GAC TGT TCA TGT GTG TTT GCA TGT-3′.

FIG. 1D shows the location of the DNA probe sequence in the predicted secondary structure of the 2,580-base mRNA (A) and the 802-base cRNA (B). The boxes show the portions of the secondary structure that is identical in both RNAs. Arrows indicate the position of the sequence complementary to the 30-mer DNA probe. Note that the probe DNA sequence corresponds to the region with no predicted secondary structure.

FIG. 3A shows the sstructure of the 9-acid dendron. Note that the 9 monomeric units form the peripheral base of the cone-shaped dendron molecule.

FIG. 3B shows the histogram of unbinding force for the interaction between the DNA probe on the 9-acid AFM tip and the 802-base cRNA on a glass slide.

FIG. 2A shows a typical force-distance curve for the interaction between the DNA probe and the 30-mer oligo RNA. Forces were recorded at a measurement rate of 0.54 μm s⁻¹.

FIG. 2B shows the histograms of the binding (Left) and unbinding (Right) forces derived from the force-distance curves of the interaction between the DNA probe and the complementary 30-mer RNA. In this example, the histogram was obtained from 191 cycles of approach and retraction. The frequency of detection for the binding and unbinding events during this process was 64%. Gaussian fitting gave the most probable force values of 32 pN and 44 pN for the binding and unbinding events, respectively.

FIG. 2C shows the histograms of the binding (Left) and unbinding (Right) forces between the DNA probe on the 9-acid dendron-modified tip and the complementary 30-mer DNA oligonucleotides on the 9-acid dendron-modified silicon substrate. The histogram was obtained from 275 cycles of approach and retraction. The frequency of detection for the binding and unbinding events during this process was 77%. Gaussian fitting gave a mean force value of 29 and 39 pN for binding and unbinding events, respectively.

FIG. 2D shows the histograms of binding (Left) and unbinding (Right) forces between the DNA probe and a non-complementary 30-mer oligo RNA. The sequence of the non-complementary RNA is 5′—NH₂(CH₂)₆-UGG GCU GAC UGU UCA UGU GUG UUU GCA UGU-3′. The histogram was obtained from 515 cycles of approach and retraction.

FIG. 4A is a schematic drawing of the experimental setup. The 802-base cRNA of the Pax6 mRNA containing the sequence complementary to the DNA probe sequence (red color, arrow) was immobilized on a glass slide.

FIG. 4B is a typical force-distance curve for the interaction between the DNA probe and cRNA. Forces were recorded at a measurement rate of 0.54 μm s⁻¹.

FIG. 4C shows the histogram of unbinding forces derived from the force-distance curves of the interaction between the DNA probe and the cRNA. The histogram was obtained from 870 cycles of approach and refraction. The frequency of detection for unbinding events during the retraction process was 73% in this example. The Gaussian fitting gave the mean value of 41+/−1 pN. The statistical error was estimated by 2σ/\sqrt{N}, where σ is the width of the distribution of the N rupture events in the histogram.

FIG. 4D shows the histograms of the unbinding forces between the DNA probe and antisense cRNA. The 802-base antisense RNA of Pax6 mRNA was synthesized in vitro and immobilized on a glass slide. The histogram was obtained from 657 cycles of approach and retraction.

FIG. 5 shows the Examples of force-distance curves for the single unbinding event between the DNA probe on the 27-acid AFM tip and the 802-base cRNA on a glass slide.

FIG. 6 shows the Examples of force-distance curves for the multiple unbinding events during the interaction of the DNA probe on the 27-acid AFM tip with the 802-base cRNA on a glass slide (A and B)

FIG. 7 shows the Examples of force histograms for the interaction between the DNA probe on the 27-acid AFM tip and the 802-base cRNA on a glass slide. Note that the most probable unbinding force on each spot ranges from 39 to 41 pN.

FIG. 8A shows the expression of the Pax6 mRNA in a coronal section of a mouse embryonic brain examined by in situ hybridization. The mRNA was detected with a digoxigenin-11-UTP-labeled anti-sense Pax6 RNA probe. Blue staining represents the presence of the labeled probe and thus expression of the Pax6 mRNA, which was detected by alkaline phosphatase-coupled anti-digoxigenin antibody. The neocortex part of the coronal section noted by a red box (Upper) is enlarged in the lower panel. Note that Pax6 mRNA was most abundant in the ventricular zone. A part of striatum where Pax6 mRNA is not expressed is marked by a blue box and used as a negative control area in (E) below.

FIG. 8B shows the maps of Pax6 mRNA distribution. Three 300 nm×300 nm areas in each of the ventricular (Upper) and cortical plate (Lower) zones were scanned. Each of the 300 nm×300 nm area was divided into 10×10 pixels for detection of the interaction force. The interaction forces were categorized into 8 levels and noted by variable colors. The number in the parenthesis indicates the number of pixels that have a mean adhesive force greater than 36 pN.

FIG. 8C shows the force maps after blocking the DNA probe-binding site in mRNA with a free competitive 30-mer DNA. The DNA sequence, 5′-TGG GCT GAC TGT TCA TGT GTG TTT GCA TGT-3′, which is the same as that of the probe DNA on the AFM tip, was incubated with the tissue section at a concentration of 40 μM for 40 min prior to measurement of force-distance curves.

FIG. 8D shows the force maps after treatment of the tissue section with RNase. The force-distance curves were recorded after incubating the tissue sample with RNase A (20 μg/ml) at 37° C. for 30 min.

FIG. 8E shows the force maps of Pax6 mRNA distribution in the striatum region of the coronal section. Note that no interaction force was larger than 33 pN.

FIG. 9 shows the Examples of force-distance curves for the single unbinding event occurring during the interaction between the DNA probe on the 27-acid AFM tip and Pax6 mRNA on a tissue section.

FIG. 10 shows the Examples of the force histograms (A) and the resulting force distribution map (B) for the interaction between the DNA probe on the 27-acid AFM tip and Pax6 mRNA on a tissue section. Note that the mean unbinding force for each pixel was then determined from the histograms of the unbinding forces recorded more than ten times at each pixel.

FIG. 11 shows the distribution of the mean unbinding force for the neocortex area (600 pixels) (A) and for the neocortex area blocked by oligo DNA complementary to the mRNA (300 pixels), in RNAse-treated tissues (300 pixels), and control striatum area (300 pixels) (B) of the tissue section as a control experiments. Note that there is no pixel for which the mean force value is between 33 and 36 pN in the neocortex area, or over 33 pN in the control experiments. The bar colors correspond to the color used in the distribution map.

FIG. 12 shows the Examples of force-distance curves for the multiple unbinding events during the interaction of the DNA probe on the 27-acid AFM tip with the Pax6 mRNA on a tissue section (A and B).

FIG. 13 shows the Examples of force histograms (A) and resulting force distribution map (B) for the interaction between the DNA probe on the 3-acid AFM tip and Pax6 mRNA on a tissue section.

FIG. 14 shows the Examples of force histograms (A) and the resulting force distribution map (B) for the interaction between the DNA probe on the 9-acid AFM tip and Pax6 mRNA on a tissue section.

### DETAILED DESCRIPTION OF THE PREFERRED EMBODIMENTS

These and other objects of the invention will be more fully understood from the following description of the invention, the referenced drawings attached hereto and the claims appended hereto.

The present invention provides a method of mapping of mRNA distribution, comprising the steps of:
(a) providing an atomic force microscopy (AFM) cantilever having a fixed end and a free end, the free end having a surface region being chemically modified by a dendron which has a plurality of termini of the branched region of the dendrons bound to the free end and an apical linear region comprising a functional group being capable of linking the dendrons to an organic moiety;
(b) preparing a probe DNA attached to the functional group of the apical liner region of the dendron on AFM cantilever where the probe DNA can specifically hybridize a target mRNA;
(c) measuring specific adhesive force between the probe DNA and the target mRNA on sectioned tissue at nanometer resolution; and
(d) identifying a molecular level distribution of the target mRNA.

The target mRNA on sectioned tissue is prepared by sectioning a sample tissue and fixing to expose the target mRNA on the surface of the tissue. The target RNA can be complementary to the probe DNA. The probe DNA is at a low density ranging about 0.01 probe/nm2 to about 0.5 probe/nm2.

The step b) is performed by deprotecting 9-anthrylmethoxycarbonyl Group of dendron, attaching NHS-group, and immobilizing the probe DNA on NHS-group.

In the present invention, the inventors utilized a DNA probe attached to a dendron-modified AFM cantilever to measure the specific adhesive force to the complementary RNA and mRNA, and to map the mRNA distribution on the surface of sectioned tissues. The probe DNA can specifically hybridize a target mRNA and is not self-complementarily.

In an embodiment of the present invention, at least a tapered protrusion is provided in the vicinity of the free end of the cantilever, and the protrusion is pyramidal or conical. Numerous analogous structures of the probe tip are used. Thus, the surface region of the free end of the cantilever is brought into contact with or into proximity with a particular protrusion so that interactions between a molecule of the reference compound and a can be measured. All types of cantilevers for AFM can be used in the present invention, and they are not specifically limited.

The cantilever may be constructed of any material known in the art for use in AFM cantilevers, including Si, SiO2, Si3N4, Si3N4Ox, Al, or piezoelectric materials. The chemical composition of the cantilever is not critical and is preferably a material that can be easily microfabricated and that has the requisite mechanical properties for use in AFM measurements. Likewise, the cantilever may be in any size and shape known in the art for AFM cantilevers. The size of the cantilever preferably ranges from about 5 microns to about 1000 microns in length, from about 1 micron to about 100 microns in width, and from about 0.04 microns to about 5 microns in thickness. Typical AFM cantilevers are about 100 microns in length, about 20 microns in width and about 0.3 microns in thickness. The fixed end of the cantilever may be adapted so that the cantilever fits or interfaces with a cantilever-holding portion of a conventional AFM.

The surface region of the free end of the cantilever may be modified for treatment with dendron for example, with siliane agents such as GPDES or TPU.

The present inventors previously demonstrated on US 2008/0113353A1 that immobilization of a DNA probe on a dendron-modified AFM tip simplifies the force-distance curves for the DNA-DNA interaction, thereby enhancing the reliability of the analysis.

Dendron is a conically shaped molecule where the repeating monomeric units are directionally stretched from a core monomer at the apex side. Thus, modification of the AFM tip surface with dendrons and subsequent attachment of a probe molecule on the apex of the dendron allows controlled spacing between the probe molecules.

The dendron may be deprotected, either in succession or in a single operation. Removal of the polypeptide and deprotection can be accomplished in a single operation by treating the substrate-bound polypeptide with a cleavage reagent, for example thianisole, water, ethanedithiol and trifluoroacetic acid.

In the addition method, the branched termini of the linear/branched polymer is attached to a suitable solid support. Suitable solid supports useful for the above synthesis are those materials which are inert to the reagents and reaction conditions of the stepwise condensation-deprotection reactions, as well as insoluble in the media used.

The removal of a protecting group such as Fmoc from the linear tip of the branched/linear polymer may be accomplished by treatment with a secondary amine, preferably piperidine. The protected portion may be introduced in about 3-fold molar excess and the coupling may be preferably carried out in DMF. The coupling agent may be without limitation O-benzotriazol-1-yl-N,N,N′,N′-tetramethyluroniumhexafluorophosphate (HBTU, 1 equiv.) and 1-hydroxy-benzotriazole (HOBT, 1 equiv.).

The dendron may be deprotected, either in succession or in a single operation. Removal of the polypeptide and deprotection can be accomplished in a single operation by treating the substrate-bound polypeptide with a cleavage reagent, for example thianisole, water, ethanedithiol and trifluoroacetic acid.

The present invention is further explained in more detail with reference to the following examples. These examples, however, should not be interpreted as limiting the scope of the present invention in any manner.

### Example 1

### Sample Preparation

### Cleaning the Substrates

Silicon wafers and fused silica plates (for dendron surface coverage analysis; data not shown) were sonicated in Piranha solution [concentrated H₂SO₄:30% H₂O₂=7:3 (v/v)) for 4 h]. The substrates were then washed thoroughly with deionized water and subsequently immersed in a mixture of deionized water, concentrated ammonia solution, and 30% hydrogen peroxide [5:1:1 (v/v/v)] in a Teflon beaker. The beaker was placed in a water bath and heated at 80° C. for 10 min. The substrates were taken out of the solution and rinsed thoroughly with deionized water. The substrates were again placed in a Teflon beaker containing a mixture of deionized water, concentrated HCl, and 30% H₂O₂ [6:1:1 (v/v/v)]. The beaker was heated at 80° C. for 10 min. The substrates were taken out of the solution and washed thoroughly with copious deionized water. The clean substrates were dried in a vacuum chamber (30-40 mTorr) for about 30 min and used immediately for the next steps.

### AFM Probe Pretreatment

Standard V-shaped silicon nitride cantilevers with pyramidal tips (MLCT-AUNM, Veeco Instruments; k=10 pN/nm) were first oxidized by dipping in 80% nitric acid and then heated at 80° C. for 20 min. The cantilevers were removed from solution and washed thoroughly with copious deionized water. The clean cantilevers were dried in a vacuum chamber (30-40 mTorr) for about 30 min and used immediately for the next steps.

### Silylation

Silicon/silica substrates and cantilevers were immersed in anhydrous toluene (20 mL) containing the silane coupling agent (0.20 mL) under a nitrogen atmosphere for 4 h. After silylation, the substrates and cantilevers were washed with toluene, and then baked for 30 min at 110° C. The substrates were immersed in toluene, toluene-methanol [1:1 (v/v)], and methanol in a sequential manner and sonicated for 3 min in each washing solution. The cantilevers were rinsed thoroughly with toluene and methanol in a sequential manner. Finally, the substrates and cantilevers were dried under vacuum (30-40 mTorr).

### Preparation of Dendron Modified Surfaces

### Preparation of 9-Acid Dendron Modified Surfaces:

The hydroxylated substrates and cantilevers were immersed for 12-24 h in a methylene chloride solution dissolving the 9-acid dendron (1.0 mM), a coupling agent, 1,3-dicyclohexylcarbodiimide (DCC) (9.9 mM), and 4-dimethylaminopyridine (DMAP) (0.9 mM). The 9-acid dendron, 9-anthrylmethyl N-({[tris({2-[({tris[(2-carboxyethoxy)methyl]methyl}amino)carbonyl]ethoxy}methyl)methyl]amino}carbonyl)propylcarbamate (or 9-acid, see Supporting Information Figure S2A) used in this work was prepared by us, and dissolved in a minimum amount of dimethylformamide (DMF) prior to adding into methylene chloride. After the reaction, the substrates were immersed in methylene chloride, methanol, and water in a sequential manner, and were sonicated for 3 min at each washing step. The cantilevers were rinsed thoroughly with methylene chloride, methanol, and water in a sequential manner. Finally the substrates and cantilevers were washed with methanol, and dried under vacuum (30-40 mTorr).

### Synthesis of the Third Generation Dendron:

### Preparation of 9-anthrylmethyl-3-({[tris({[tris[2-tris{[2-methoxycarbonyl)ethoxy]-methyl}methyl)amino]carbonyl}ethoxy)methyl]methyl}amino)carbonyl]-2-ethoxy}methyl)methyl]amino}carbonyl)propylcarbamate (or 27-ester)

The second generation/9-acid dendron, 9-anthrylmethyl-3-({[tris({2-[({tris[(2-carboxyethoxy)methyl]methyl}amino)carbonyl]ethoxy}methyl)methyl]amino}-carbonyl)propylcarbamate (or 9-acid), was prepared as described previously (B. J. Hong et al., Langmuir 21, 4257, 2005.). The 9-acid (0.5 g, 0.31 mM, 1.0 equiv), 1-[3-(dimethylamino)-propyl]-3-ethylcarbodiimide hydrochloride (EDC, 0.59 g, 3.1 mM, 10 equiv), and 1-hydroxybenzotriazole hydrate (HOBT, 0.42 g, 3.1 mM, 10 equiv) were dissolved in methylene chloride and stirred at room temperature. Tris[((methoxycarbonyl)ethoxy)methyl]-aminomethane (1.1 g, 2.9 mM, 9.3 equiv) dissolved in methylene chloride was added with stirring. After stirring at room temperature for 36 h, the methylene chloride was evaporated. The crude product was dissolved in ethyl acetate (200 ml) and sequentially washed with 10% HCl, water, 10% aqueous Na₂CO₃, saturated aqueous NaHCO₃ and brine. After drying with anhydrous MgSO₄, filtering, and evaporating, the resultant viscous yellow liquid was dried under vacuum. The total weight of crude yellow liquid was 1.5 g, which was hydrolyzed without further purification.

### Preparation of 9-anthrylmethyl-3-({[tris({[(1-tris[(2-{[tris{[2-carboxyethoxy]methyl}methyl)amino]carbonyl}ethoxy)methyl]methyl}amino)carbonyl]-2-ethoxy}methyl)methyl]amino}carbonyl)propylcarbamate (or 27-acid)

The crude 9-anthrylmethyl-3-({[tris({[(1-{tris[(2-{[(tris {[2-(methoxycarbonyl)ethoxy]methyl}methyl)amino]carbonyl}ethoxy)methyl]methyl}amino)carbonyl]-2-ethoxy}methyl)methyl]amino}carbonyl)propylcarbamate (or 27-ester, 1.5 g) obtained above was dissolved in acetone (75 ml) and 0.40 N NaOH (75 ml). After stirring at room temperature for 1 day, the acetone was evaporated. The aqueous solution was washed with ethyl acetate, stirred in an ice bath and acidified with aqueous 10% HCl. After the product was extracted with ethyl acetate, the organic solution was dried with anhydrous MgSO₄, filtered, and evaporated. The total weight of final yellow powder was 1.1 g (Y=79%).

¹H NMR (DMSO-d₆): δ 13.00-11.00 (br, CH₂COOH, 27H), 8.67 (s, C₁₄H₉CH₂, 1H), 8.42 (d, C₁₄H₉CH₂, 2H), 8.14 (C₁₄H₉CH₂, 2H), 7.62 (t, C₁₄H₉CH₂, 2H), 7.54 (t, C₁₄H₉CH₂, 2H), 6.97 (t, OCONHCH₂, 1H), 6.85 (s, OCH₂CH₂CONHC, 3H), 6.82 (s, OCH₂CH₂CONHC, 9H), 6.80 (s, CH₂CH₂CH₂CONHC, 1H), 6.06 (s, C₁₄H₉CH₂O, 2H), 3.55 (m, CH₂OCH₂CH₂CONH, CH₂OCH₂CH₂COOH, 156H), 3.02 (q, NHCH₂CH₂, 2H), 2.42 (t, CH₂CH₂COOH, 54H), 2.32 (t, OCH₂CH₂CONH, 24H), 2.11 (t, CH₂CH₂CH₂CONH, 2H), 1.59 (m, CH₂CH₂CH₂, 2H)

¹³C NNMR (DMSO-d₆): δ 172.6 (CH₂COOH), 170.4 (OCH₂CH₂CONH), 170.2 (CH₂CH₂CH₂CONH), 156.3 (OCONH), 130.9 (C₁₄H₉CH₂), 130.4 (C₁₄H₉CH₂), 128.8 (C₁₄H₉CH₂), 127.4 (C₁₄H₉CH₂), 126.6 (C₁₄H₉CH₂), 125.2 (C₁₄H₉CH₂), 124.9 (C₁₄H₉CH₂), 124.2 (C₁₄H₉CH₂), 68.2 (NHCCH₂OCH₂CH₂COOH), 67.3 (NHCCH₂OCH₂CH₂CONH), 67.0 (NHCCH₂OCH₂CH₂CONH), 66.6 (NHCCH₂OCH₂CH₂COOH), 59.6 (C₁₄H₉CH₂), 59.4 (NHCCH₂O), 36.3 (NHCH₂CH₂CH₂CONH), 34.5 (NHCCH₂OCH₂CH₂), 30.4 (NHCH₂CH₂CH₂CONH), 25.1 (CH₂CH₂CH₂)

### Preparation of 27-Acid Dendron Modified Surfaces:

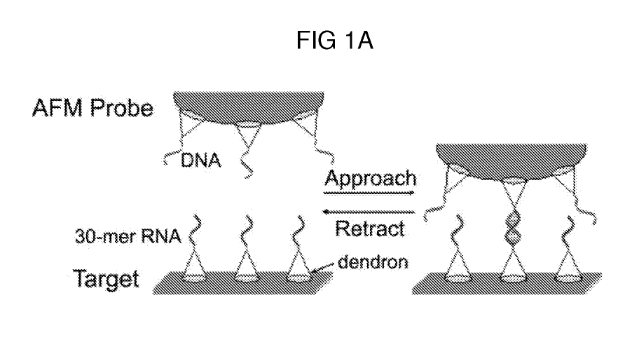

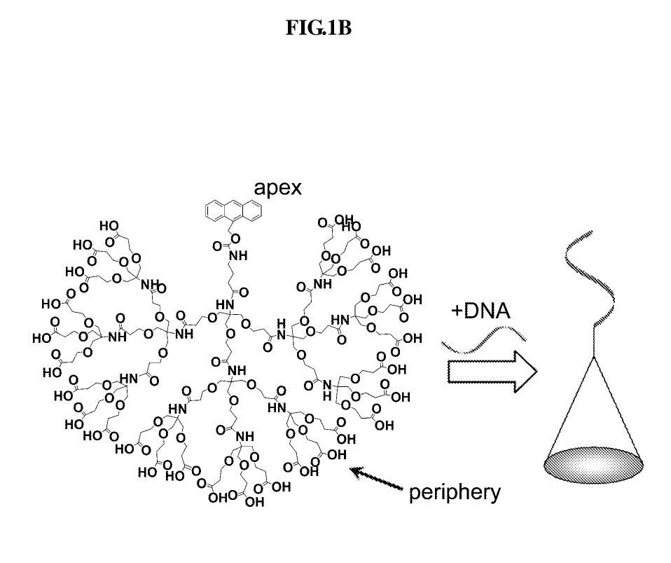

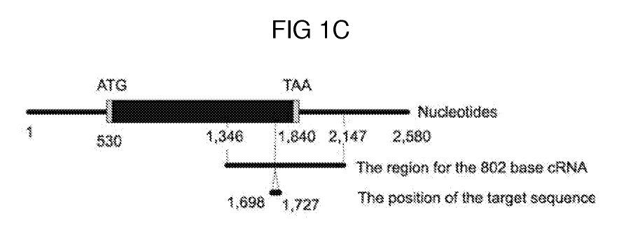

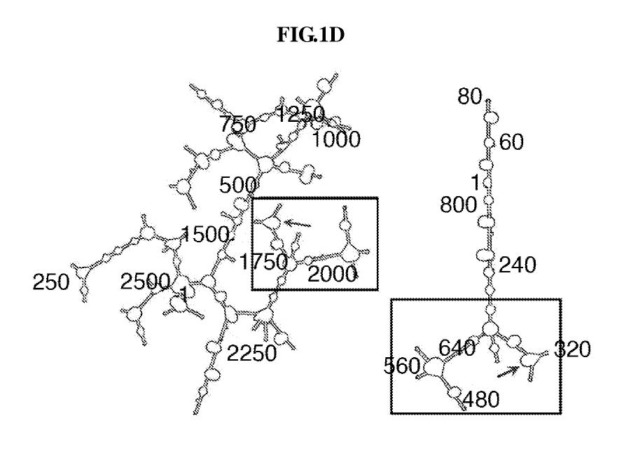

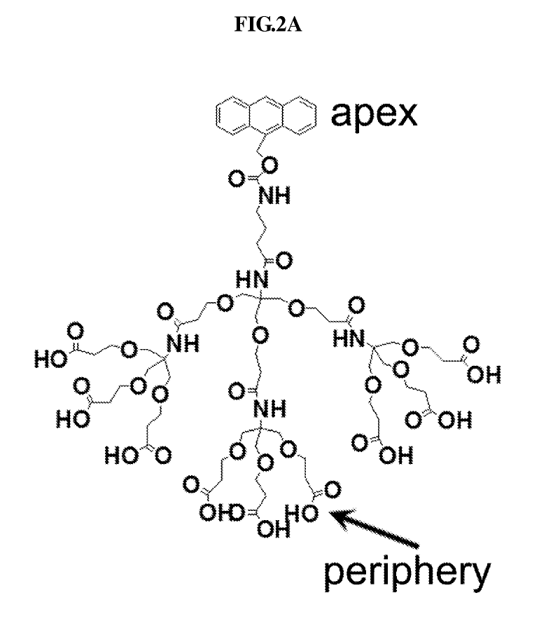

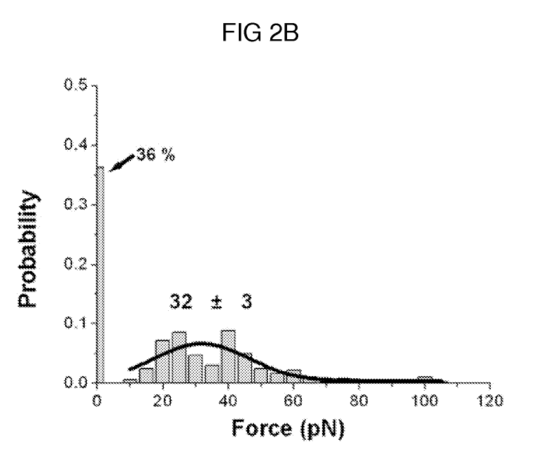

The above hydroxylated substrates and cantilevers were immersed for 12-24 h in a methylene chloride solution dissolving the 27-acid dendron (1.0 mM), a coupling agent, 1,3-dicyclohexylcarbodiimide (DCC) (29.7 mM), and 4-dimethylaminopyridine (DMAP) (2.9 mM). The 27-acid dendron, 9-anthrylmethyl-3-({[tris({[(1-{tris[(2-{[tris{[2-carboxyethoxy]methyl}methyl)amino]carbonyl}ethoxy)methyl]methyl}amino)carbonyl]-2-ethoxy}methyl)methyl]amino}carbonyl)propylcarbamate (or 27-acid, see FIG. 1A) used in this work was prepared by us (see Supporting Information), and dissolved in a minimum amount of dimethylformamide (DMF) prior to adding into methylene chloride. After the reaction, the substrates were immersed in methylene chloride, methanol, and water in a sequential manner, and were sonicated for 3 min at each washing step. The cantilevers were rinsed thoroughly with methylene chloride, methanol, and water in a sequential manner. Finally the substrates and cantilevers were washed with methanol, and dried under vacuum (30-40 mTorr).

Deprotection of the 9-anthrylmethoxycarbonyl Group

The cantilevers and dendron-modified substrates were stirred for 2 h in a methylene chloride solution containing trifluoroacetic acid (TFA) (1.0 M). After the reaction, they were soaked in a methylene chloride solution with 20% (v/v) diisopropylethylamine (DIPEA) for 10 min. The substrates were sonicated in methylene chloride and methanol each for 3 min, and the cantilevers were rinsed thoroughly with methylene chloride and methanol in a sequential manner. The substrates and cantilevers were dried under vacuum (30-40 mTorr).

### Preparing NHS-Modified Substrates

The above deprotected substrates and cantilevers were immersed for 4 h under nitrogen in an acetonitrile solution containing di(N-succinimidyl)carbonate (DSC) (25 mM) and DIPEA (1.0 mM). After the reaction, the substrates and cantilevers were placed in stirred DMF for 30 min and washed with methanol. The substrates and cantilevers were dried under vacuum (30-40 mTorr).

### Immobilization of DNA/Isolated Short RNA

The above NHS-modified substrates were placed in a solution containing 30-mer RNA [20 μM in 25 mM NaHCO₃ buffer (pH 8.5) with 5.0 mM MgCl₂] for 12 h. In parallel, the NHS-modified cantilevers were placed in a solution of 30-mer DNA [20 μM in 25 mM NaHCO₃ buffer (pH 8.5) with 5.0 mM MgCl₂] for 12 h. The sequence of the 30-mer RNA is 5′—NH₂(CH₂)₆-ACA UGC AAA CAC ACA UGA ACA GUC AGC CCA-3′(SEQ ID NO: 1), and its complementary 30-mer DNA sequence is 5′—NH₂(CH₂)₆-TGG GCT GAC TGT TCA TGT GTG TTT GCA TGT-3(SEQ ID NO:2)', of which GC content is 47%. After the reaction, the substrates and cantilevers were stirred in a buffer solution [2×SSPE buffer (pH 7.4) containing 7.0 mM sodium dodecylsulfate] at 37° C. for 1 h, and were rinsed thoroughly with water to remove non-specifically bound oligonucleotides. Finally, the substrates and cantilevers were dried under vacuum (30-40 mTorr).

### Example 2

### The Interaction Force Between DNA and 30-mer RNA (Model System I)

### 2-1: Sample Preparation

To measure the interaction force between the DNA probe immobilized on the AFM tip and the 30-mer oligo RNA complementary to the DNA probe on the silicon wafer (FIG. 1A), DNA probe and 30-mer oligo RNA on the AFM tip and silicon wafer were immobilized using the so-called 9-acid dendron and 27-acid dendron (FIG. 1B). The AFM tip and surface modification of the substrate were performed according to Examples 1.

The DNA probe used was a 30-mer oligonucleotide complementary to nucleotides 1698-1727 of Pax6 mRNA (FIG. 1C and FIG. 1D). The DNA probe with an amine group at the 5′ side was covalently linked to the apex of a dendron immobilized on the AFM tip. The target RNA with an amine group at the 5′ position was attached to the apex of an immobilized dendron on the surface of a silicon wafer.

### 2-2: AFM Force Measurement

All force measurements were performed with a NanoWizard AFM (JPK Instrument). The spring constant of each AFM tip was calibrated in solution before each experiment by the thermal fluctuation method. The spring constants of the cantilevers employed varied between 10-15 pN/nm. All measurements were carried out in fresh PBS buffer (pH 7.4) at room temperature. All force measurements were recorded with a measurement velocity of 0.54 μm s⁻¹. To measure the mean force values, the force-distance curves were always recorded more than 100 times at one position on a substrate, and more than 2 spots were examined in each separate experiment.

### 2-3: Results

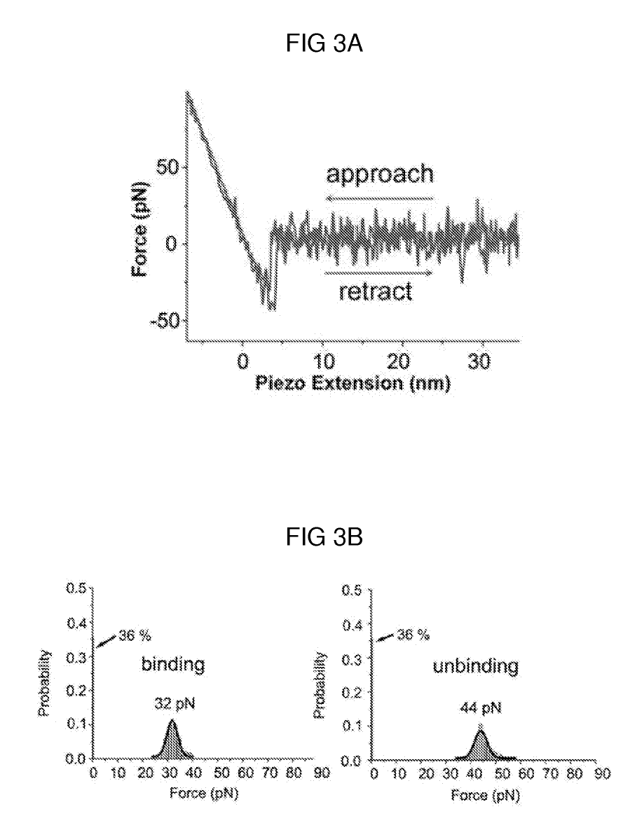

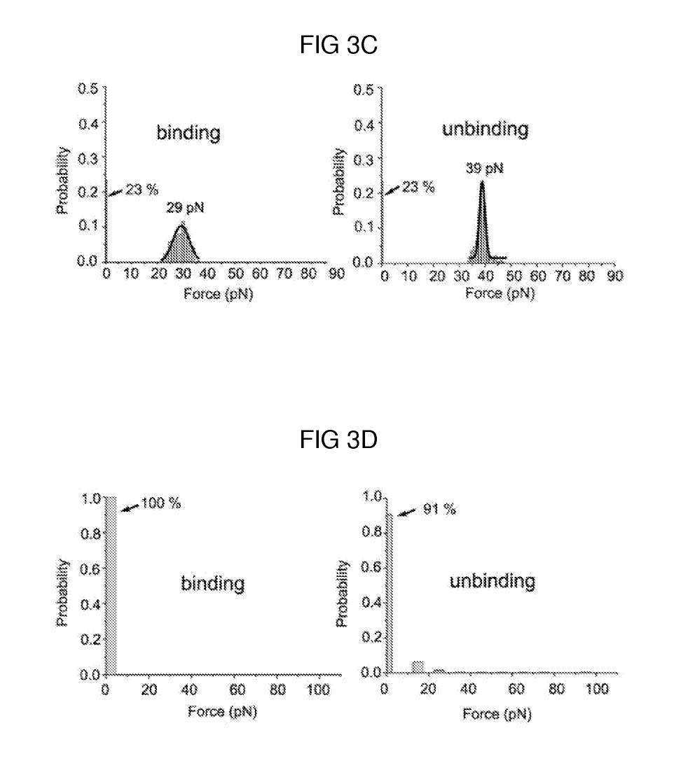

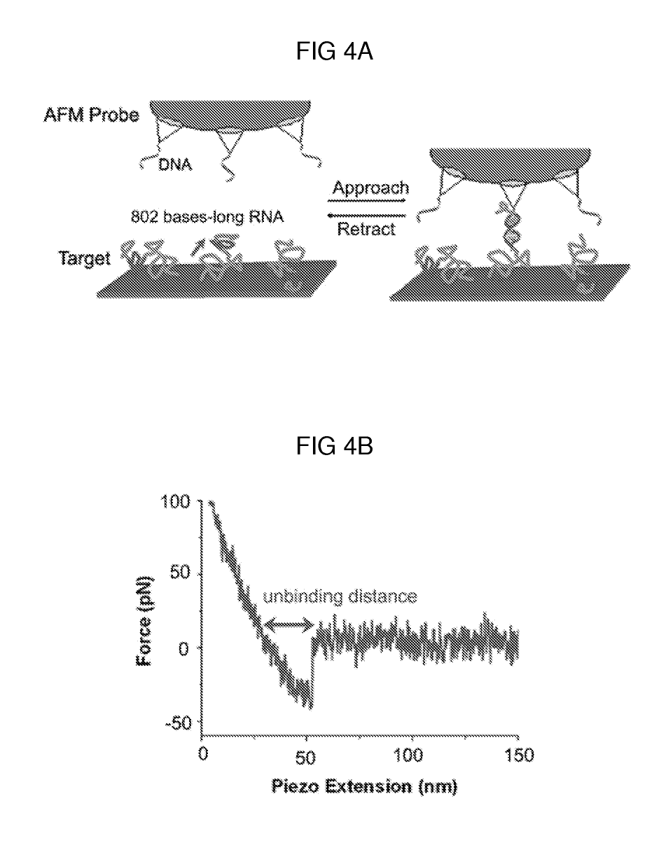

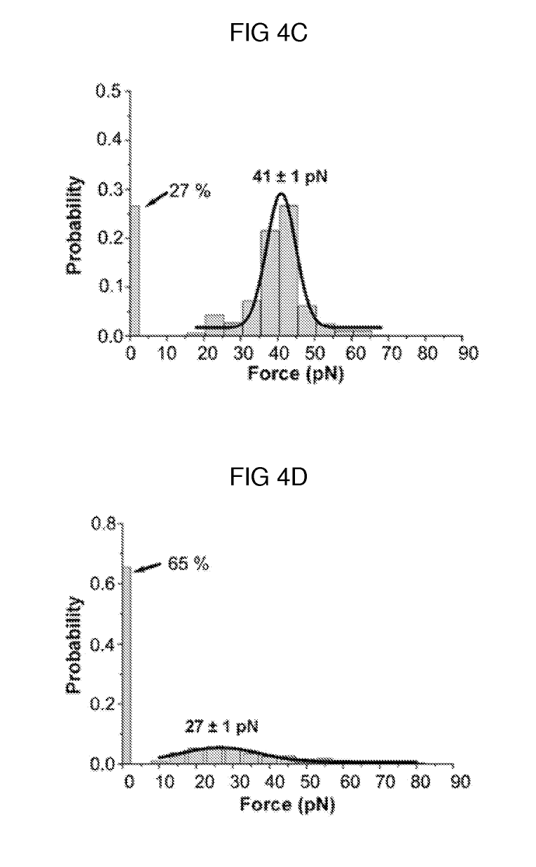

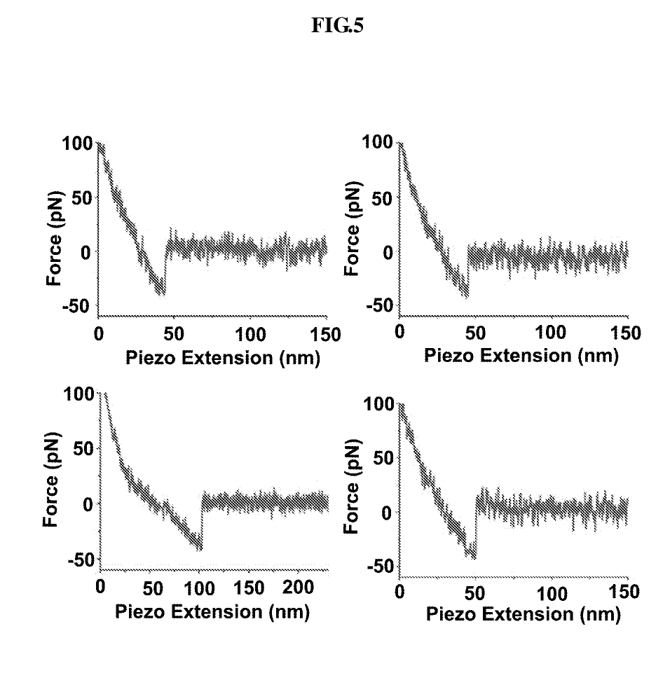

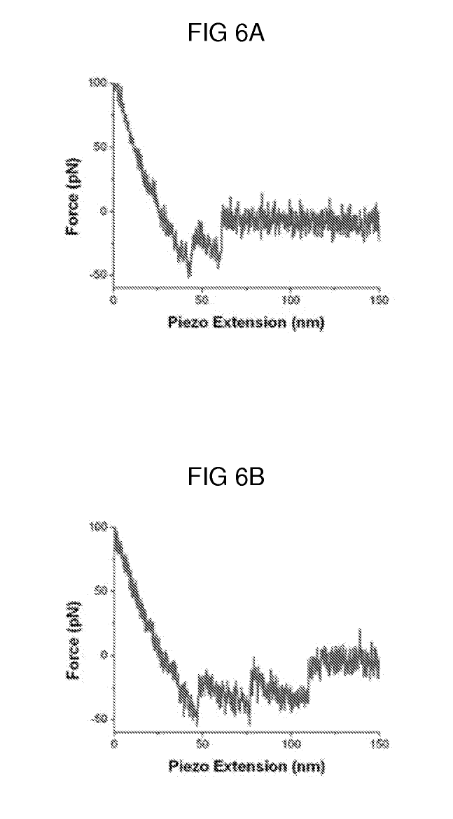

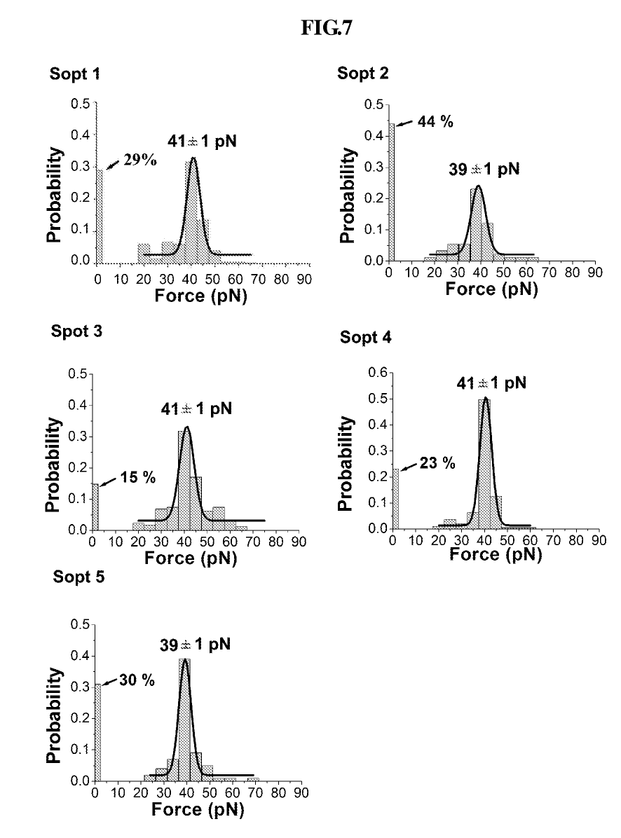

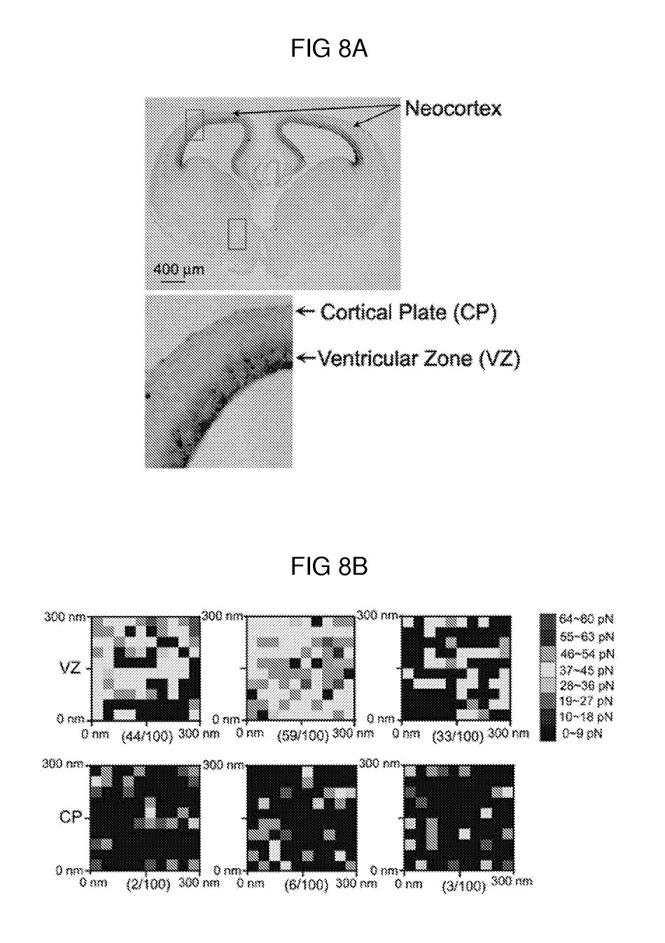

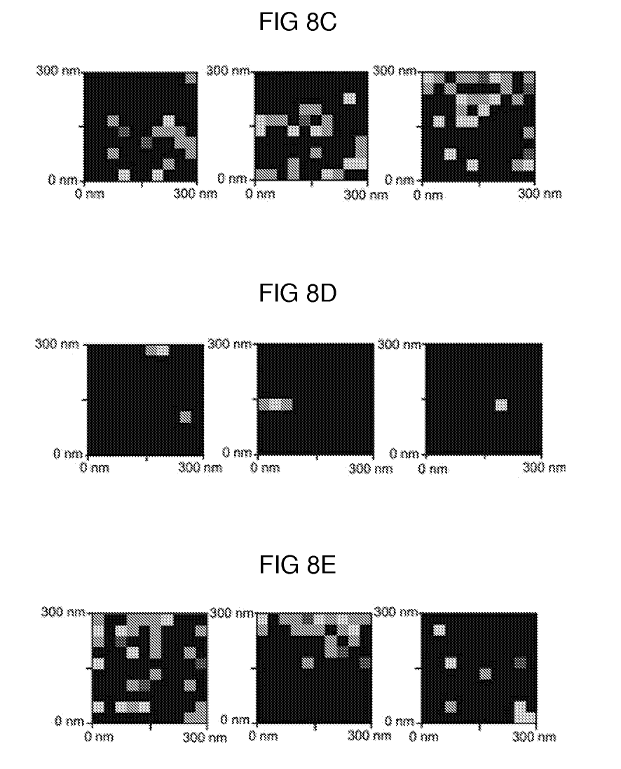

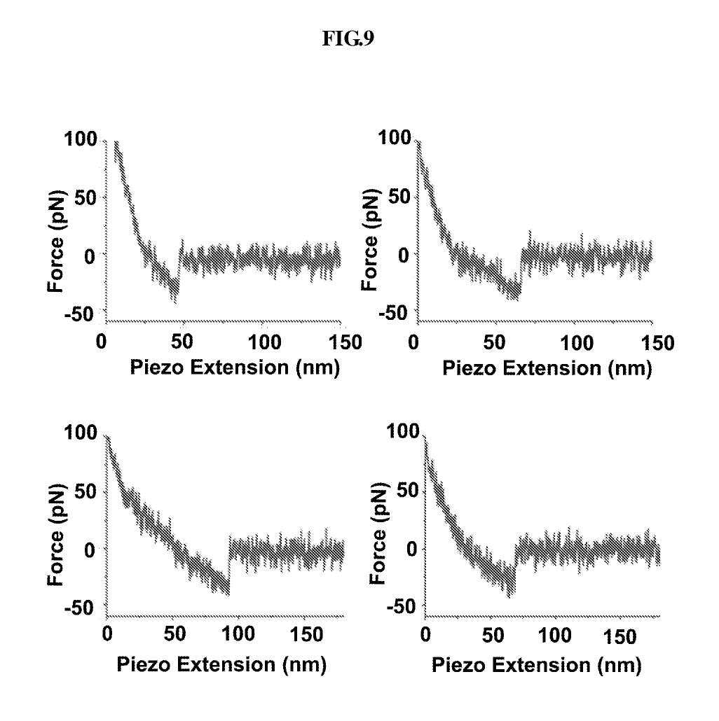

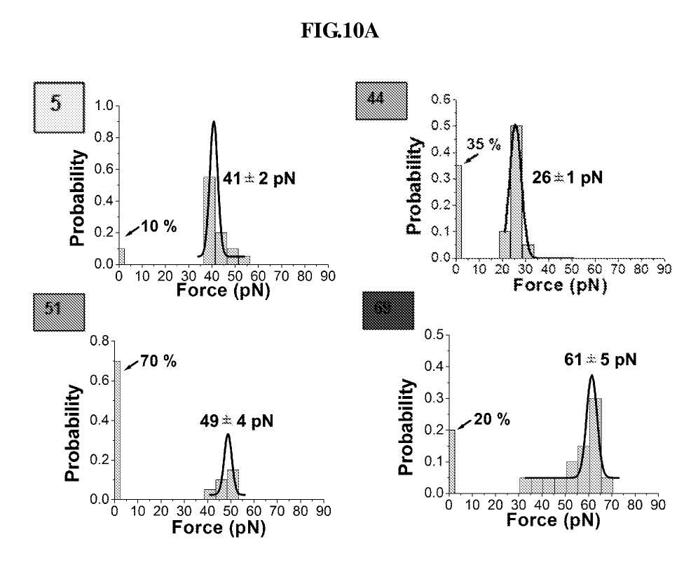

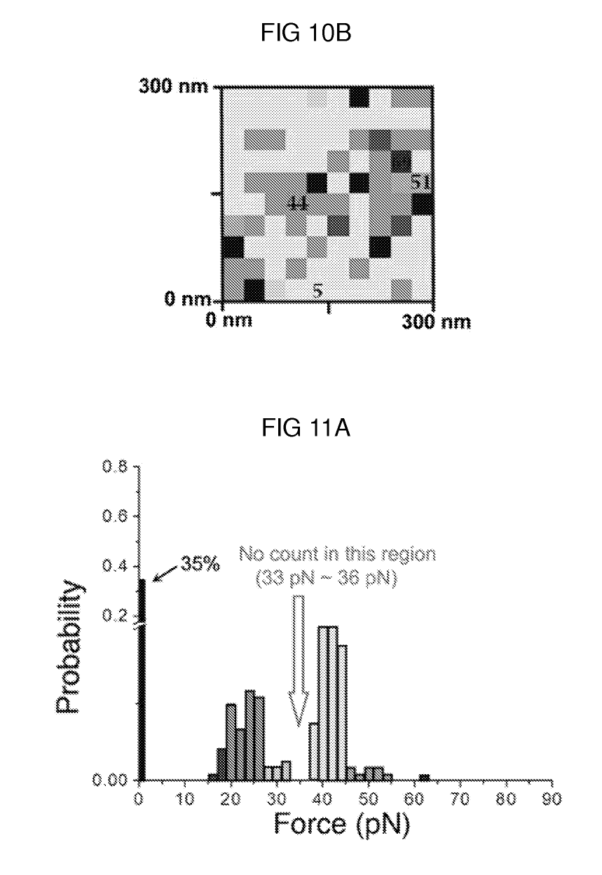

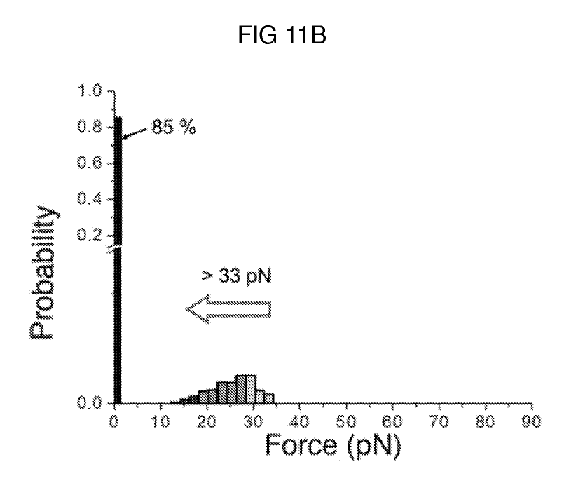

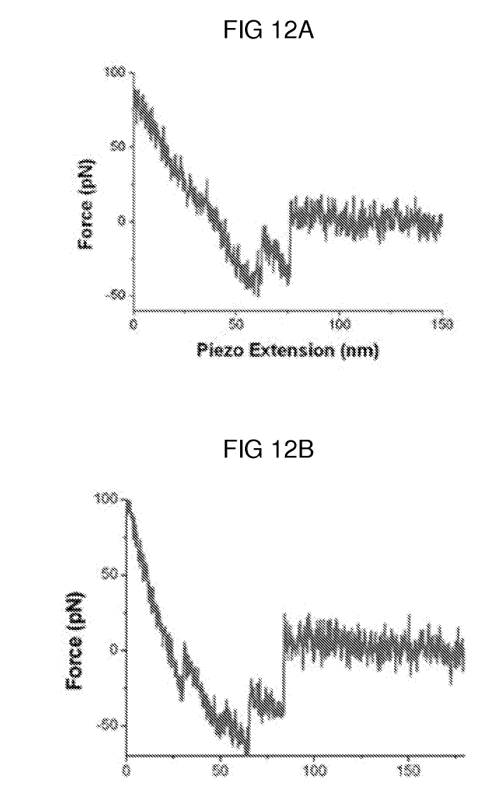

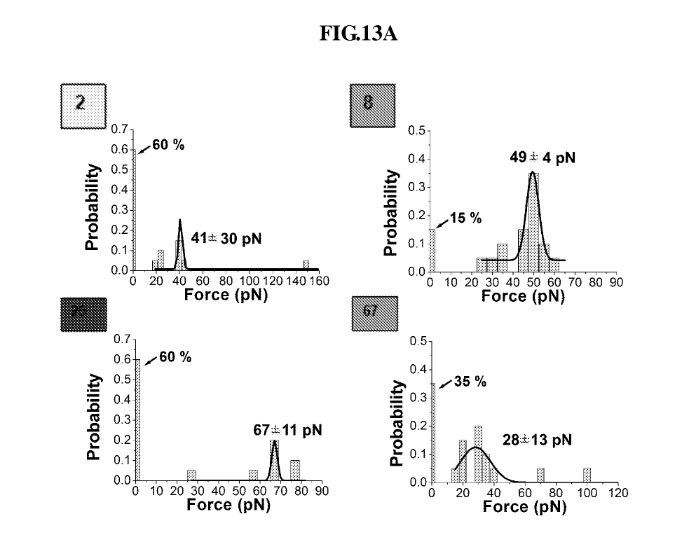

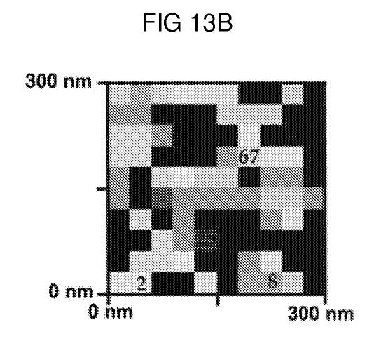

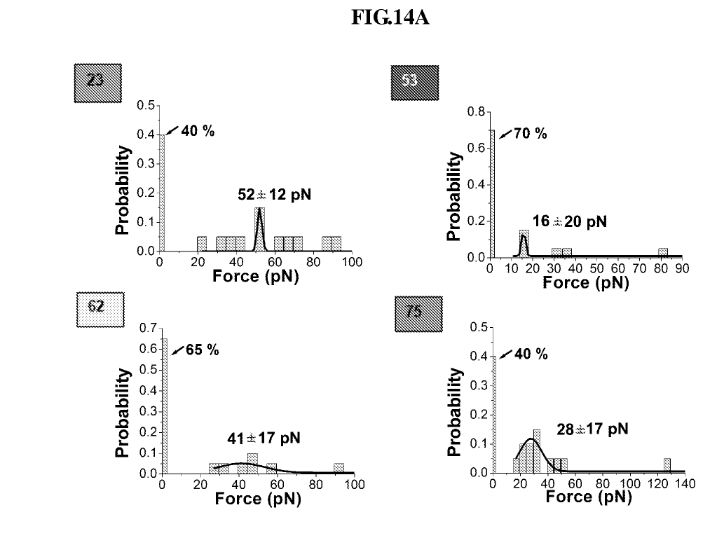

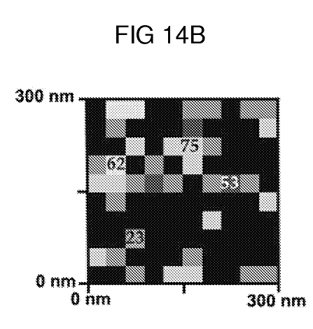

The present inventors initially employed the so-called 9-acid dendron. The 9-acid dendron led to satisfactory measurement of DNA-DNA interaction forces in previous study (Y. J. Jung et al., J. Am. Chem. Soc. 129, 9349, 2007); conjugation between the 9 carboxylic acids in the periphery of the dendron (FIG. 2A) and the AFM tip surface provided mesospacing for the DNA probes attached to the apex. While the DNA probe on the 9-acid dendron-modified AFM tip properly detected the complementary target oligo-RNA molecule, it yielded an unsatisfactorily broad force distribution histogram for a longer target RNA (FIG. 2B). Employment of a 27-acid dendron (FIG. 1B) that provided larger spacing between the DNA probes on the apex yielded an interaction force histogram satisfactorily narrow for analysis of the DNA-RNA interactions. Here, the present inventors describe our analysis of the interaction force mapping of mRNA with the 27-acid dendron-modified AFM tip. The present inventors obtained force-distance curves of the interaction between the DNA probe and target RNA during the approach and retraction processes, respectively. The resulting force-distance curves showed a single distinctive binding (attractive) and unbinding (adhesive) pattern during the approach and retraction processes, respectively (FIG. 3A). Force curves demonstrated linear unbinding profiles prior to unbinding rupture and the distance (binding and unbinding) in the force curves varied in the range of 3-7 nm. Binding and unbinding forces were obtained from each force-distance curve to generate force histograms (FIG. 3B). Gaussian fitting of these histograms yielded mean forces of 32 and 44 pN for the binding and unbinding events, respectively. In this experiment, the binding and unbinding forces were same as those measured with the 9-acid dendron-modified AFM tip (data not shown).

It is known that an RNA-DNA duplex is more stable than the corresponding DNA-DNA interaction (S. M. Freier et al., Proc. Natl. Acad. Sci. U.S.A. 83, 9373, 1986). When the corresponding DNA-DNA interaction was measured using a 9-acid dendron-modified AFM tip and silicon substrate and a 0.54 μm s⁻¹ measurement rate (FIG. 3C). In order to avoid the error that might occur during the calibration process, the present inventors employed an identical tip for the comparison in this particular experiment. It was possible to use one tip for both experiments because the only thing we had to change was the substrate. The interaction binding and unbinding forces were smaller than those of the DNA-RNA interaction by 3 and 5 pN, respectively. To verify the specificity, the interactions between the probe DNA and non-complementary target RNA were measured. Binding was not observed, the unbinding force was significantly less than the specific mean force, and the unbinding frequency was dramatically reduced (FIG. 3D).

### Example 3

### The Interaction Forces between DNA and Partial-Length Pax6 RNA of 802 Bases (Model System II)

### 3-1: Sample Preparation

To examine the interaction force between the AFM tip-bound DNA probe and its complementary RNA sequence residing in a long RNA molecule (FIG. 4A), the DNA probe and 802-base RNA on the AFM tip and glass slide were immobilized using the 27-acid dendron. The AFM tip was performed according to Examples 1.

The 802-base RNA (cRNA) corresponding to nucleotides 1,346-2,147 of Pax6 mRNA (FIG. 1C) was synthesized in vitro. The method of synthesis of 802-base RNA was as follows.

Synthesis of the 802-Base cRNA for Pax6 mRNA

The cDNA corresponding to the nucleotide sequence from 1,346 and 2,147 of mouse Pax6 mRNA was amplified by PCR from a mouse cDNA library, using two primers (5′-TCTAATCGAAGGGCCAAATG-3′ (SEQ ID NO:3) and 5′-TCCAACAGCCTGTGTTGTTC-3′(SEQ ID NO:4); the former corresponds to the nucleotide sequence from 1,346 to 1,365 and the latter from 2,128 to 2,147). The sequence information for mouse cDNAs was obtained from a database (GeneBank accession no. NM 013627). This 802-bases PCR product was cloned into a pGEM-T vector (Promega, USA). The resulting pGEM-PAX6 vector was linearized with KspI and Not I and was used as a template to synthesize the 802-base sense and antisense RNA of Pax6 mRNA, respectively, performing an in vitro transcription using SP6/T7 transcription kits (Roche Diagnostics, Germany). After transcription, the cDNA template was removed with RNase-free DNase I. The remaining RNA solution was adjusted to 0.4 M LiCl and centrifuged for precipitation of RNA. The final concentration of RNA was quantified by UV spectrometry. This cRNA, which included nucleotides 1698-1727 complementary to the probe DNA sequence, was fixed on a glass slide as follows.

Immobilization of the 802-Base cRNA on a Glass Slide

The cRNA was adjusted to a concentration of 0.5 μg/μl in 150 mM sodium phosphate buffer (pH 7.4). Using a gel loading pipette tip, 1.0 μl of the RNA solution was loaded onto glass slides (ProbeOn Plus, Fisher Scientific, USA) and was left to dry at room temperature for 30 min, which lead to a typical spot diameter of 5 mm. To immobilize the RNA on the surface of the glass slide, the slide was heated in an oven at 65° C. for 30 min and was subsequently irradiated with UV light (120 mJ) for 2 min 40 s with a UV Stratalinker (Stratagene, USA). The RNA-bound glass slide was then incubated in a blocking buffer solution [50% formamide, 10% dextran sulfate, 250 μg/ml yeast tRNA, 0.3 M NaCl, 20 mM Tris-HCl (pH 8.0), 5 mM EDTA, 10 mM sodium phosphate, 1% sarcosyl, 0.1% bovine serum albumin, 0.1% ficoll, 0.1% polyvinylpyrollidone] at 65° C. for 1 h. The slides were washed by dipping in PBS buffer five times (each time for 10 min) and finally in water for a few seconds before air-drying. For force-measurements, the air-dried slide was rehydrated in PBS buffer.

### 3-2: AFM Force Measurement

All force measurements were performed with a NanoWizard AFM (JPK Instrument). The spring constant of each AFM tip was calibrated in solution before each experiment by the thermal fluctuation method. The spring constants of the cantilevers employed varied between 10-15 pN/nm. All measurements were carried out in fresh PBS buffer (pH 7.4) at room temperature. All force measurements were recorded with a measurement velocity of 0.54 μm s⁻¹. To measure the mean force values, the force-distance curves were always recorded more than 100 times at one position on a substrate, and more than 2 spots were examined in each separate experiment.

### 3-3: Results

Binding and unbinding force curves between the 30-mer DNA probe on the dendron-modified tip and cRNA on the slide were obtained. Force-distance curve patterns during the retraction process indicated mostly single (FIG. 4B and FIG. 5) and double (FIG. 6A) unbinding events with a rarity of more than two unbinding events (FIG. 6B). The multiple unbinding events might be explained by poor control of the surface density of 802-base cRNA and secondary interactions with neighboring cRNA. The unbinding forces for multiple unbinding events calculated from the last rupture event were incorporated into the force histogram. These mean unbinding forces between the DNA probe and the cRNA from each spot ranged from 39-41 pN (FIG. 4C and FIG. 7). The unbinding probability from each spot is different each other because the surface fixation method results in randomly oriented 802 bases RNA on surface. The orientation of 802 bases RNA on the glass slide should give minimal influence on the mean unbinding force. The only thing that would change is the probability of recording the interaction. The observed forces were from specific interactions between the DNA probe and cRNA sequence, since the force histogram for interactions with the antisense sequence cRNA was quite different, with a reduced mean unbinding force (27 pN) and frequency of unbinding events (FIG. 4D). The mean unbinding force of the 802-base cRNA (39-41 pN) was slightly less than that of the 30-mer oligo RNA (44 pN).

### 3-4: Discussion

In case of an 802 bases cRNA, interestingly, no distinctive binding events were recorded in the force-distance curve of the interaction between the DNA probe and cRNA. The absence of the binding event could be explained by the fact that the DNA binding site is located at the inner part of 802 base-long cRNA with which exists in the form of complicated secondary structures (FIG. 4A). In addition, force curves demonstrated nonlinear unbinding profiles prior to unbinding rupture (FIG. 4B, FIG. 5, and FIG. 6), whereas those of the 30-mer RNA manifested linear profiles (FIG. 3A). This difference was likely due to the enhanced flexibility of the 802-base cRNA, which should result in a reduced actual loading rate during unbinding (Y. Gilbert et al., Nano Lett. 7, 796, 2007). In particular, the unbinding distance in the force curves of 802-base cRNA, varied in the range of 8-40 nm, while that of the 30-mer oligo RNA varied in the range of 3-7 nm. The rupture distance of 802-base cRNA reflect the rupture distance of 30-mer RNA and the forced extension of a certain parts of 802-base cRNA fixed on the solid surface. Force-distance curve patterns during the refraction process of 802-base cRNA indicated mostly single (FIG. 4B and FIG. 5) and double (FIG. 6A) unbinding events with a rarity of more than two unbinding events (FIG. 6B). The multiple unbinding events might be explained by poor control of the surface density of 802-base cRNA and secondary interactions with neighboring cRNA.

### Example 4

### Mapping of Pax6 mRNA on a Mouse Embryonic Tissue

### 4-1: Sample Preparation

To map the distribution in mouse E14.0 embryonic brain tissue, the DNA probe was immobilized using the 27-acid dendron according to Examples 1. The mouse embryonic tissue was prepared as follows.

### Preparation of Mouse Embryonic Tissue Sections

Brains from C57BL/6 mouse embryos at E14.0 were dissected in PBS buffer and fixed with gentle rocking for 12 h in 4% paraformaldehyde (PFA) at 4° C. Plug date was defined as embryonic day 0.5 (E0.5). The brain tissue was then washed in PBS buffer with 4% PFA and rinsed briefly in an embedding medium (Tissue-Tec, USA). The brain tissues in the embedding medium were fast-frozen in isopentane cooled with liquid nitrogen. Serial coronal sections of 12 μm thickness were prepared with a freezing microtome and were collected on glass slides (ProbeOn plus, Fisher Scientific, USA). Tissue sections were fixed with 4% PFA in PBS buffer for 10 min and rinsed with PBS solution before treatment with Proteinase K (4 μg/ml) in PBS buffer for 8 min at room temperature. Tissue sections were post-fixed with 4% PFA, rinsed with PBS, dehydrated sequentially in 70% and 95% ethanol for a few seconds before air-drying. The in situ hybridization of the embryonic tissue sections was basically performed according to a standard protocol (B.-K. Koo et al., Development 132, 3459, 2005).

### 4-2: AFM Force Measurement

All force measurements were performed with a NanoWizard AFM (JPK Instrument). The spring constant of each AFM tip was calibrated in solution before each experiment by the thermal fluctuation method. The spring constants of the cantilevers employed varied between 10-15 pN/nm. All measurements were carried out in fresh PBS buffer (pH 7.4) at room temperature. All force measurements were recorded with a measurement velocity of 0.54 μm s⁻¹. Force images were obtained by processing the force values recorded during the raster-scanning on areas of 300 nm×300 nm each. The area was divided by 10×10 pixels. Concerning statistics and stochastic behaviour, force-distance curves were typically recorded more than ten times at each pixel, and the presented force value of each pixel is the mean unbinding force from fitting the force distribution to a Gaussian curve.

### 4-3: Results

The present inventors mapped the distribution in mouse E14.0 embryonic brain tissue, when Pax6 mRNA is expressed in the ventricular and subventricular zones (N. Warren et al., Cereb. Cortex 9, 627, 1999; C. Englund et al., J. Neurosci. 25, 247, 2005; R. F. Hevner et al., Neurosci. Res. 55, 223, 2006). The brain tissue was sectioned and fixed to expose mRNA on its surface. The present inventors confirmed Pax6 mRNA expression in the neocortical region by in situ hybridization with a digoxigenin-labeled antisense Pax6 RNA probe. Consistent with previous reports (N. Warren et al., Cereb. Cortex 9, 627, 1999; C. Englund et al., J. Neurosci. 25, 247, 2005; R. F. Hevner et al., Neurosci. Res. 55, 223, 2006), Pax6 mRNA was much more abundant along the ventricular zone than along the cortical plate side (FIG. 8A). The ventricular zone and cortical plate sides were then subjected to mRNA mapping, employing the same conditions used nucleotides for detection of cRNA immobilized on glass. Pax6 mRNA mapping was performed by detecting the specific unbinding force between the DNA probe and Pax6 mRNA on the sectioned tissue (FIG. 8B). In each mapping area, 10×10 pixels were examined to obtain force-distance curves for the unbinding processes. Representative force distance curves for single rupture unbinding events are shown in FIG. 9. The mean unbinding force value for each pixel was determined from the histograms of the unbinding forces (FIG. 10) recorded more than ten times. The mean unbinding force of each pixel was displayed as a force map by categorizing the force values into 8 levels (FIG. 8B), with a reference point at 37 pN, as the mean unbinding force distribution of each pixel showed a distinctive trough between 33 and 36 pN (FIG. 11A). Of the 600 pixels examined, no single pixel showed the value in this range. We considered any force over 37 pN to be derived from the specific interaction between the DNA probe and Pax6 mRNA, while the reason for the deviation from 41 pN observed in 802 bases RNA was not clear, complication in tissue sample should be one of the reasons for this deviation. As the controls, no force over 33 pN was observed in tissues blocked by oligo DNA complementary to the mRNA (FIG. 8C), in RNAse-treated tissues (FIG. 8D), or in the control area of the tissue sections (FIG. 8E). The largest forces observed in these cases were 33, 31, and 33 pN (FIG. 11B), respectively.

### 4-4: Discussion

In case of a fixed mouse embryonic brain tissue, interestingly, no distinctive binding events were recorded in the force-distance curve of the interaction between the DNA probe and cRNA. The absence of the binding event could be explained by the fact that the DNA binding site is located at the inner part of Pax6 mRNA with which exists in the form of complicated secondary structures. In addition, force curves demonstrated nonlinear unbinding profiles prior to unbinding rupture (FIG. 9, and FIG. 12), whereas those of the 30-mer RNA manifested linear profiles (FIG. 3A). This difference was likely due to the enhanced flexibility of the Pax6 mRNA, which should result in a reduced actual loading rate during unbinding (Y. Gilbert et al., Nano Lett. 7, 796, 2007). In particular, the unbinding distance in the force curves of Pax6 mRNA, varied in the range of 8-40 nm, while that of the 30-mer oligo-RNA varied in the range of 3-7 nm. The rupture distance of Pax6 mRNA reflects the rupture distance of 30-mer RNA and the forced extension of a certain parts of Pax6 RNA embedded in the tissue. Force-distance curve patterns during the retraction process indicated single (FIG. 9) and double (FIG. 12A) unbinding events with more than two unbinding events (FIG. 12B). Notably, the tissue sample demonstrated more frequent multiple unbinding events than the cRNA samples. Other neighboring mRNAs of which sequences are partially identical to that of the targeted RNA might cause the multiple rupture events.

Force maps indicated that Pax6 mRNA was detected at a much higher frequency in the ventricular zone than in the cortical plate zone. In the force map of FIG. 8B, the numbers of the positive pixels were 136 and 11 for the ventricular and cortical plate zones, respectively, out of 300 pixels examined. Thus, the frequency of positive pixels in the two zones was proportional to Pax6 mRNA levels detected by in situ hybridization, supporting the concept that these force maps reflect the local distribution of Pax6 mRNA in a given area of tissue.

From several control experiments including the force maps in the control area of the tissue sections of FIG. 8A, presence of Pax6 mRNA in cortical plate was clearly confirmed. Because the probe DNA detects only the mRNA molecules appeared on the surface of the examined tissue that are accessible to the probe, it is difficult to suggest that the number of the pixels with a certain force value is linearly dependent to the mRNA on surface of the examined tissue. Also, the hydrodynamic length of the probe DNA would allow sensing mRNA at the neighboring pixel. Nevertheless, it is expected that most of mRNA molecules present at the tissue surface were sensed because the orientation of mRNA at the surface would affect minimally the most probable adhesive force, while the probability of recording an interaction would be influenced.

Therefore, it is believed that the force maps recorded by picoforce AFM correctly shows the trend of the mRNA distribution. It is important to note the high sensitivity of the employed approach enables the detection of the mRNA in the ventricular zone, while the Pax6 protein has not been detected by the fluorescence assay in the section. The presence and location of mRNA molecules in a sectioned tissue can be facilely detected using a DNA probe attached to a dendron-modified AFM tip. This mRNA detection procedure is straightforward once the DNA probe is properly selected and immobilized on a suitably modified AFM tip. The choice of dendron in AFM tip modification was a critical factor. The use of a 27-acid dendron led to successful detection of the mRNA, whereas tips modified with a lower generation dendron (3-acid or 9-acid) led to unsatisfactory results, with frequent nonspecific and multiple rupture events and broad force histograms (FIG. 13 and FIG. 14).

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1-5. (canceled)

6. A method for mapping mRNA distribution on a sectioned tissue sample, said method comprising:
scanning the sectioned tissue sample with an atomic force microscope (AFM) comprising a probe DNA that is capable of selectively hybridizing to a target mRNA; and
determining the level of target mRNA distribution within the sectioned tissue sample using the adhesive force measured with the atomic force microscope,
wherein the probe DNA is attached to an AFM cantilever via a dendron comprising:
a plurality of termini that are attached to the surface of the AFM cantilever; and
a single apical linear region that is attached to the probe DNA.

7. The method of claim 6, wherein said step of scanning the sectioned tissue sample with the AFM comprises measuring adhesive force at nanometer resolution.

8. The method of claim 6, wherein the dendron comprises twenty seven (27) termini.

9. The method of claim 8, wherein the dendron is of the formula:

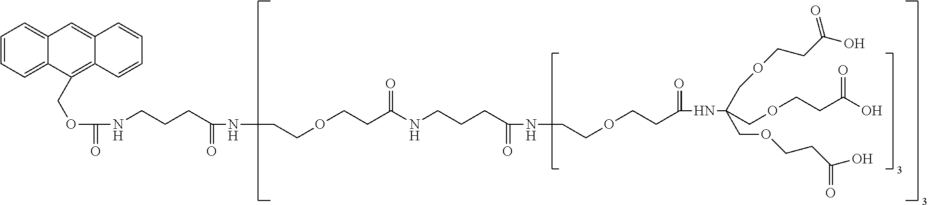

10. The method of claim 6, wherein the target mRNA on sectioned tissue sample is prepared by sectioning a sample tissue and fixing the section sample tissue to expose the target mRNA on the surface of the sectioned tissue sample.

11. The method of claim 6, wherein the density of the probe DNA attached to the AFM cantilever ranges from about 0.01 probe/nm² to about 0.5 probe/nm².
